(19) United States
(12) Patent Application Publication
(10) Pub. No.: US 2025/0064877 A1
(43) Pub. Date: Feb. 27, 2025

(54) COMPOSITION FOR INCREASING MUSCLE STRENGTH CONTAINING COMPLEX EXTRACT OF ANGELICA KEISKEI AND LEAVES OF MORUS ALBA L.

(71) Applicant: HAMSOA PHARMACEUTICAL CO., LTD., Seoul (KR)

(72) Inventors: Hyug Yong CHOI, Seoul (KR); Hyun Joo CHO, Seoul (KR); Youn Seon KIM, Gyeonggi-do (KR); Tae Hee KIM, Gyeongsangnam-do (KR); Jae Hyun YOON, Jeollabuk-do (KR); Yung Hyun CHOI, Busan (KR); Seon Yeong JI, Busan (KR); Hyun HWANGBO, Busan (KR); Da Hye KIM, Busan (KR); Min Yeong KIM, Busan (KR)

(73) Assignee: HAMSOA PHARMACEUTICAL CO., LTD., Seoul (KR)

(21) Appl. No.: 18/720,663

(22) PCT Filed: Jul. 25, 2023
(86) PCT No.: PCT/KR2023/010710

§ 371 Date: Jun. 17, 2024

(30) Foreign Application Priority Data
Oct. 14, 2022 (KR) 10-2022-0132202

(51) Int. Cl. A61K 36/232; A61K 31/12; A61K 31/216; A61K 31/352; A61K 31/7048; A61K 36/605; A61P 21/00; A61P 39/06

(52) U.S. Cl. CPC A61K 36/232; A61K 31/12; A61K 31/216; A61K 31/352; A61K 31/7048; A61K 36/605; A61P 21/00; A61P 39/06; A61K 2236/333

(57) ABSTRACT

Proposed is a muscle strength-enhancing composition being capable of maintaining or strengthening muscles or preventing muscle-related diseases. More specifically, proposed is a food composition containing a composite extract as an active ingredient obtained by mixing Angelica keiskei and Morus alba L. at a weight ratio of 1:8 to 16 and extracting the resulting mixture with alcohol. The food composition promotes antioxidant activity (for example, DPPH scavenging activity and ABTS scavenging activity) and muscle function enhancement activity which suppresses muscle protein breakdown and promotes muscle building, whereby the food composition prevents muscle-related diseases by enabling muscles to be maintained or strengthened.

### TECHNICAL FIELD

The present disclosure relates to a muscle strength-enhancing composition containing a mixture of extracts from ashitaba (Angelica keiskei) and leaves of mulberry (Morus alba L.).

### BACKGROUND ART

Sarcopenia refers to the loss of muscle mass and the loss of function due to aging. Deterioration of muscle function induces disease and aging in the body, ultimately resulting in social problems in the long term. Exercise and nutrition are known factors that affect muscle loss. According to several studies, protein intake and muscle mass are related. In particular, in the case of growing adolescents, a lack of activity and exercise may affect muscle development and growth.

Muscle function depends primarily on the formation of muscle cells and the increase in muscle mass. A lack of exercise causes the breakdown of muscle proteins and direct muscle loss. Muscle is a tissue expressed from mesoderm stem cells, accounts for approximately 40% to 50% of body weight, and is responsible for maintaining movement while a person is alive. In general, muscle refers to skeletal muscle, which is made from a collection of muscle fibers formed through muscle cell differentiation. The number of muscle fibers and the level of muscle mass within the muscle are the source of muscle strength.

Differentiation into muscle cells is mainly active during the growth/development period, and after this period, muscle growth occurs through the maintenance and regeneration process of skeletal muscle, so muscle formation/development during the growth period is very important. Additionally, muscle loss is caused by decreases in exercise amount, protein intake, and metabolic capability. Herein, the decrease in exercise amount involves aging, which affects the damage and breakdown of the muscle fibers that make up muscles. Therefore, it is important to control muscle loss as well as to develop muscle during the growth period and later to build muscle.

In addition, muscle health gradually gains attention due to the increased occurrence of obesity and chronic wasting diseases, as well as an aging population. Because of this, ‘exercise-mimicking medicine for antiaging’ was selected as one of the promising future candidate technologies. The exercise-mimicking medicine can prevent geriatric diseases such as muscle aging by presenting exercise effects to a person even without actual exercise. Concerning this, the development of materials has been actively underway, the materials being capable of helping maintain muscles even in various environments where muscles may decrease or atrophy. In particular, to discover new materials from natural products that are safe and show excellent pharmacological efficacy, much research has been conducted.

These muscle function enhancement materials should ultimately enhance muscle strength through increased muscle mass. Thus, it is important for the materials to practically show effectiveness in both stimulating muscle development and suppressing muscle loss. After researching this, the present inventors confirmed that the composite extract of Angelica keiskei and leaves of mulberry promoted superior antioxidant activity and muscle function enhancement activity to the single extract of each of the two materials, and as a result, completed the present disclosure.

### DISCLOSURE

### Technical Problem

The technical task of the present disclosure is to provide a muscle strength-enhancing composition containing a composite extract of Angelica keiskei and leaves of mulberry.

### Technical Solution

### To Fulfill the Technical Task,

The present disclosure provides a muscle strength-enhancing food composition containing a composite extract of Angelica keiskei and leaves of mulberry as an active ingredient. The composite extract is obtained by mixing Angelica keiskei and the leaves of mulberry at a weight ratio of 1:8 to 16 and extracting the resulting mixture with alcohol.

The food composition promotes antioxidant activity (for example, DPPH scavenging activity and ABTS scavenging activity) and muscle function enhancement activity which suppresses muscle protein breakdown and promotes muscle building, whereby the food composition prevents muscle-related diseases by enabling a muscle to be maintained or strengthened.

According to the present disclosure, the muscle-related diseases include one or more selected from the group consisting of atony, muscular atrophy, muscular dystrophy, muscle degeneration, muscle rigidity, atrophic axonal sclerosis, myasthenia gravis, cachexia, and sarcopenia.

According to the present disclosure, the composite extract contains xanthoangelol or 4-hydroxyderricin derived from Angelica keiskei as an active ingredient and contains quercetin, quercetin-3-O-malonyl glucoside, or chlorogenic acid derived from the leaves of mulberry as an active ingredient.

### Advantageous Effects

A muscle strength-enhancing food composition containing a composite extract of Angelica keiskei and leaves of mulberry of the present disclosure contains a composite extract as an active ingredient, the composite extract being obtained by extracting the mixture of Angelica keiskei and the leaves of mulberry with alcohol. Accordingly, the food composition shows its effectiveness in promoting antioxidant activity (for example, DPPH scavenging activity and ABTS scavenging activity) and muscle function enhancement activity which suppresses muscle protein breakdown and promotes muscle building. Therefore, the food composition of the present disclosure is safe as a natural product-derived material and can be applied to muscle strengthening, muscle enhancement, muscle differentiation, muscle regeneration, and muscle loss treatment or can be applied to prevention, improvement, or treatment of muscle fatigue. In addition, the food composition can help maintain or strengthen muscles even in various situations that induce muscle atrophy, so the food composition can be effectively applied to prevent muscle-related diseases.

### DESCRIPTION OF DRAWINGS

FIG. 1 is a graph showing the DPPH radical scavenging abilities of a composite of leaves of mulberry and Angelica keiskei according to an embodiment of the present disclosure;

FIG. 2 is a graph showing the ABTS cation radical scavenging abilities of a composite of leaves of mulberry and Angelica keiskei according to another embodiment of the present disclosure;

FIG. 3 is a schematic diagram of a muscle atrophy animal experiment process according to yet another embodiment of the present disclosure;

FIG. 4 is a graph showing changes in body weight and grip strength in an animal model of muscular dystrophy according to yet another embodiment of the present disclosure;

FIG. 5 is a graph showing changes in soleus muscle thickness and weight in an animal model of muscular dystrophy according to yet another embodiment of the present disclosure;

FIG. 6 is a graph showing histological changes in animal models of muscular dystrophy according to yet another embodiment of the present disclosure; and

FIG. 7 is a graph showing changes in the expression of MyoD and MuRF1 in animal models of muscular dystrophy according to yet another embodiment of the present disclosure.

### MODE FOR DISCLOSURE

Since the present disclosure may help to modify the technology in various ways and have various embodiments, specific embodiments will be illustrated in the drawings and described in detail. However, this is not intended to limit the present disclosure to specific embodiments and should be understood to include all changes, equivalents, and substitutes included in the spirit and technical scope of the present disclosure. While describing each drawing, similar reference numerals are used for similar components.

Throughout this specification and claims, unless otherwise specified, the terms ‘comprise’, ‘comprises’, and ‘comprising’ are used to indicate the presence of features, numbers, steps, reactions, elements, or combinations thereof described in the specification. However, the terms should be understood that the terms do not exclude in advance the possibility of the presence or addition of one or more other features, numbers, steps, reactions, components, or combinations thereof.

The terms used in the present disclosure are only used to describe specific embodiments and are not intended to limit the present disclosure. Singular expressions include plural expressions unless the context clearly dictates otherwise.

Unless otherwise defined, all terms used herein, including technical or scientific terms, have the same meaning as commonly understood by a person of ordinary skill in the technical field to which the present disclosure pertains. Terms defined in commonly used dictionaries should be interpreted as having meanings consistent with the meanings they have in the context of the relevant technology. Unless explicitly defined in this application, the terms are not to be construed in an idealized or overly formal sense.

The present disclosure relates to a muscle strength-enhancing food composition containing a composite extract of Angelica keiskei and leaves of mulberry as an active ingredient. The composite extract is obtained by mixing Angelica keiskei and the leaves of mulberry at a weight ratio of 1:8 to 16, extracting the resulting mixture with alcohol. The food composition promotes antioxidant activity (for example, DPPH scavenging activity and ABTS scavenging activity) and muscle function enhancement activity which suppresses muscle protein breakdown and promotes muscle building, whereby the food composition prevents muscle-related diseases by enhancing muscle strength maintained or strengthened.

The food composition of the present disclosure promotes antioxidant activity. ‘Antioxidation’ refers to suppressing cellular oxidation caused by highly reactive free radicals or reactive oxygen species (ROSs), the generation of which is induced by oxidative stresses caused by intracellular metabolism or the effects of ultraviolet rays. The term has a concept that includes a reduction in cell damage by removing the free radicals or reactive oxygen species. The antioxidant activity is evaluated by DPPH radical scavenging activity or ABTS cation radical scavenging activity, which involves the scavenging of free radicals. According to a preferred embodiment of the present disclosure, the single extract of the leaves of mulberry promoted low DPPH radical scavenging activity and ABTS cation radical scavenging activity. The composite extract promoted higher activity than the single extract of Angelica keiskei. In particular, the composite extract promoted superior activity in both DPPH radical scavenging activity and ABTS cation radical scavenging activity to the single extract of Angelica keiskei, despite the composite extract having a lower content of Angelica keiskei than the single extract. The mixing ratio of each single extract was particularly important. Excellent activity was shown when the extract of Angelica keiskei and the extract of the leaves of mulberry were mixed in a ratio of 1:1 to 2:1, and in particular, the best activity was shown when the extracts were mixed at a ratio of 2:1.

The composite extract is an alcohol extract obtained by extracting Angelica keiskei and the leaves of mulberry with alcohol. The composite extract contains xanthoangelol or 4-hydroxyderricin derived from Angelica keiskei as an active ingredient and also contains quercetin, quercetin-3-O-malonyl glucoside, or chlorogenic acid derived from the leaves of mulberry as an active ingredient. According to another preferred embodiment of the present disclosure, in the case of Angelica keiskei, both xanthoangelol and 4-hydroxyderricin were detected in the test solution, so it was confirmed that the materials might be set as indicator ingredients. In the case of the leaves of mulberry, it was confirmed that quercetin 3-O-malonylglucoside and chlorogenic acid among quercetin, quercetin 3-O-malonylglucoside, and chlorogenic acid might be set as indicator ingredients. In addition, it was confirmed that the content of the active ingredient, which was Xanthoangelol, was hundreds of times higher when alcohol extraction of Angelica keiskei was conducted compared to hot water extraction. In the case of the extract of the leaves of mulberry, it was confirmed that the content of the active ingredient, which was quercetin 3-O-malonylglucoside, was 26 to 27 times higher when alcohol extraction was conducted compared to hot water extraction. Therefore, more preferably, the composite extract contains xanthoangelol and quercetin 3-O-malonylglucoside as active ingredients.

In addition, to obtain the satisfactory content of the active ingredients for the composite extract through the extraction of raw materials, after the extraction of each raw material, it is necessary to consider the yield and content of solids that may be obtained from the raw materials, and the extraction process. According to yet another preferred embodiment of the present disclosure, the yields of obtained concentrate powder, relative to the input of the raw materials, was 14% for Angelica keiskei and 3.5% for the leaves of mulberry, which meant that the yield in the case of Angelica keiskei was 4 times higher than that in the case of the leaves of mulberry From there, it was confirmed that the raw materials included active ingredients, and it was necessary to mix the raw materials considering each yield to excellently promote activity. Accordingly, for the composite extract to promote excellent antioxidant activity compared to a single extract of each raw material, the extract of Angelica keiskei and the extract of the leaves of mulberry should be mixed in a ratio of 1:1 to 2:1. The food composition preferably contains a composite extract obtained by mixing Angelica keiskei and the leaves of mulberry at a weight ratio of 1:8 to 16 and extracting the resulting mixture with alcohol. Herein, the alcohol-extracted composite extract preferably contains at least 0.5 mg/g of xanthoangelol and at least 5.0 mg/g of quercetin 3-O-malonylglucoside obtained by optimizing the yield and extraction process.

In addition, dexamethasone is a synthetic glucocorticoid that is used to treat inflammation, allergies, autoimmune diseases, and cancer, but dexamethasone is a substance widely used in muscle atrophy-related research since dexamethasone is known to cause muscle atrophy by promoting the breakdown of proteins related to muscle mass. This occurs when dexamethasone is administered over a long period of time. Additionally, muscle atrophy is related to two muscle-specific E3 ubiquitin ligases, MAFbx (muscle atrophy F-box)/atrogin-1 and MuRF1 (Muscle RING Finger-1). Dexamethasone decomposes muscle proteins and decreases myogenin and MyoD (myogenic differentiation antigen), which are muscle-specific factors involved in muscle differentiation, resulting in a decrease in muscle mass.

According to yet another embodiment of the present disclosure, in animal models of muscle atrophy induced by dexamethasone, when measuring the body weight of the animal models, the body weight was confirmed to be maintained similar to that in the control group administered only sterilized distilled water. When measuring grip strength, the actual muscle function of the animal models was maintained. In other words, the composite extract of the present disclosure promoted muscle function enhancement activity that suppressed muscle loss and allowed muscles to be maintained even in an environment where muscles might decrease or atrophy.

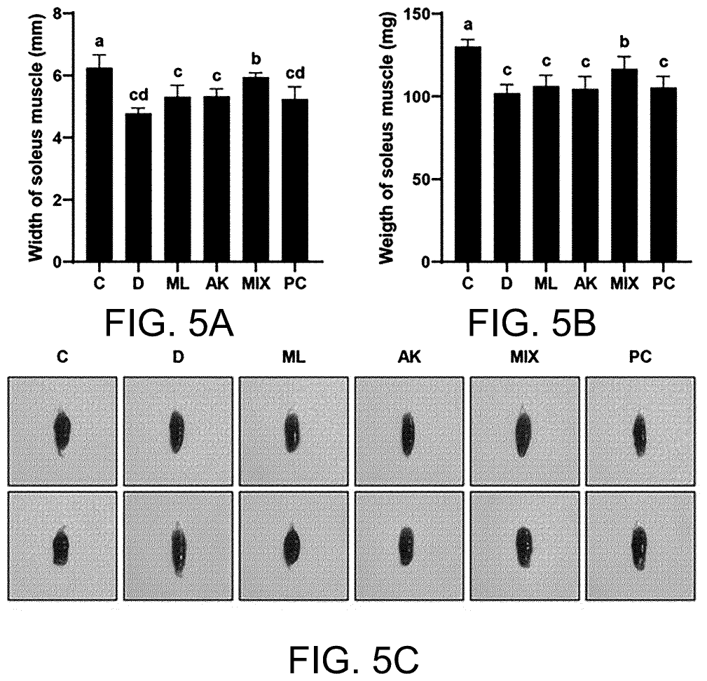

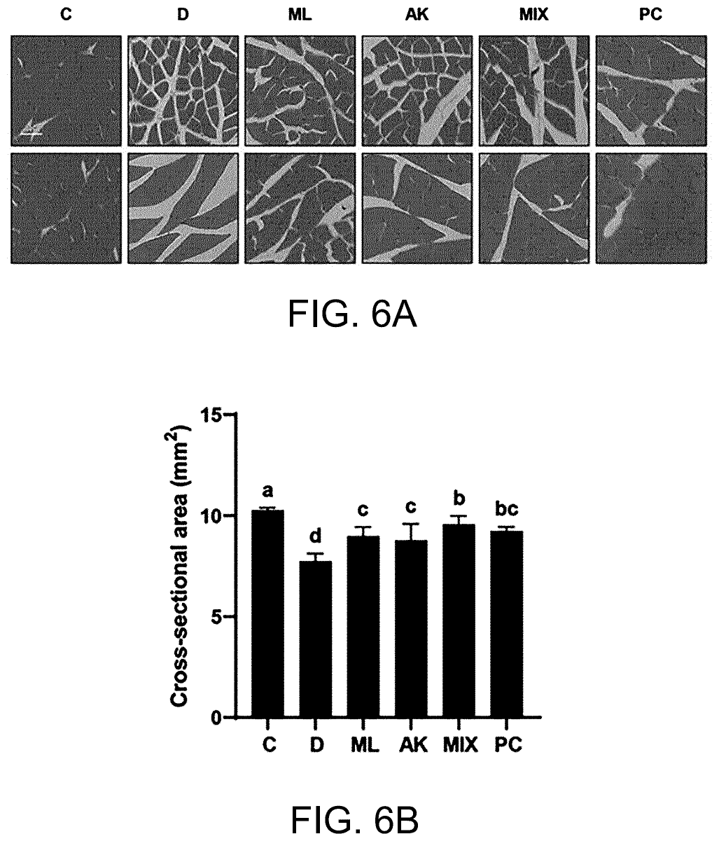

In addition, when examining changes in the thickness and weight of the soleus muscle (FIG. 5), it was confirmed that the thickness and weight of the soleus muscle significantly decreased in the dexamethasone-induced group whereas the thickness and weight of the soleus muscle significantly increased in the composite extract-administered group. However, when confirming the histological changes in the gastrocnemius muscle (FIG. 6), the muscle fibers were atrophied, resulting in a decrease in the size of the muscle fibers and the widening gap between muscle fibers in the induced muscle. Meanwhile, the atrophy of muscle fibers was suppressed in the composite extract-administered group. The atrophy level was the most similar to that of the control group. In other words, the composite extract of the present disclosure maintained and strengthened muscles even in a muscle atrophy environment through muscle function enhancement activity.

In addition, when confirming the effect of the leaves of mulberry and Angelica keiskei on the expression of MyoD (a factor involved in muscle production in the process of treating muscle atrophy), the expression of MyoD was decreased by dexamethasone while the expression was partially increased in the single extract-administered group. In addition, the expression of MyoD was recovered to a similar extent in the composite extract-administered group to that in the control group. It was confirmed that the composite extract of the present disclosure promoted muscle function enhancement activity that promotes muscle production.

In addition, when confirming the effect on the expression of MuRF1 (a factor related to muscle atrophy), the expression of MuRF1, which was involved in decomposing muscle proteins, was increased in the dexamethasone-induced group and significantly decreased in the composite extract-administered group. In other words, the composite extract of the present disclosure promoted muscle function enhancement activity that might prevent muscle loss by regulating the mechanism that suppressed muscle protein breakdown.

As such, the composition of the present disclosure promotes the muscle function enhancement activity by suppressing muscle protein decomposition and promoting muscle production with the containment of a composite extract of the leaves of mulberry and Angelica keiskei as active ingredients. Thus, the composition may prevent muscle-related diseases by maintaining or strengthening muscles. Herein, the muscle-related diseases include one or more selected from the group consisting of atony, muscular atrophy, muscular dystrophy, muscle degeneration, muscle rigidity, atrophic axonal sclerosis, myasthenia gravis, cachexia, and sarcopenia.

The food composition containing the composite extract of Angelica keiskei and the leaves of mulberry of the present disclosure may be used as it is or in combination with other foods or food ingredients. The food composition may be appropriately included according to conventional methods to provide a health functional food.

The health functional food composition is preferably prepared in any one formulation selected from the group consisting of powder, granules, pills, tablets, capsules, candies, syrup, and beverages, but the formulation is not limited thereto. The health functional food composition of the present disclosure may be prepared by adding the active ingredients as they are or mixing the active ingredients with other foods or food ingredients. The health functional food composition may be appropriately prepared according to conventional methods. The health functional food composition may include various nutrients, vitamins, minerals (electrolytes), synthetic and natural flavors, colorants and enhancers (cheese and chocolate), pectic acid and salts thereof, alginic acid and salts thereof, organic acids, and protective colloidal thickeners, pH adjusters, stabilizers, preservatives, glycerin, alcohol, and carbonating agents used in carbonated beverages. Additionally, the health functional food composition may include pulp for the production of natural fruit juice and vegetable drinks. The ingredients may be used independently or in combination. In addition, the health functional food composition of the present disclosure may include various flavoring agents or natural carbohydrates as additional ingredients. The natural carbohydrates include monosaccharides (for example, glucose and fructose), disaccharides (for example, maltose and sucrose), polysaccharides (for example, dextrin and cyclodextrin), and sugar alcohols (for example, xylitol, sorbitol, and erythritol). As sweeteners, natural sweeteners (for example, thaumatin and stevia extract) and synthetic sweeteners (for example, saccharin and aspartame) may be used.

Hereinafter, the present disclosure will be described in detail through examples. However, the following examples are only examples to aid understanding of the present disclosure, and the scope of the present disclosure is not limited thereto.

The examples of the present disclosure may be combined with any other examples unless clearly indicated to the contrary. Any feature indicated as being particularly preferred or advantageous may be combined with any other feature or feature indicated as being preferred or advantageous.

### <Example 1> Preparation of Individual Extract Powder and Measurement of Indicator Ingredient Content

### 1-1 Establishment of Extraction Conditions

(1) The results of experiments with Angelica keiskei and leaves of mulberry were confirmed to identify indicator ingredients. In the case of Angelica keiskei, both xanthoangelol and 4-hydroxyderricin were detected in the test solution, and it was confirmed that the materials might be set as indicator ingredients. In the case of the leaves of mulberry, it was confirmed that quercetin 3-O-malonylglucoside and chlorogenic acid among quercetin, quercetin 3-O-malonylglucoside, and chlorogenic acid might be set as indicator ingredients.

(2) To set optimal extraction conditions (temperature, solvent, and time) for Angelica keiskei and the leaves of mulberry, the extraction of each of Angelica keiskei and the leaves of mulberry was performed under three conditions: hot water extraction at a temperature of 110° C., 40% alcohol, and a temperature of 55° C. The content of indicator ingredients was measured for each extraction time. Herein, among the indicator ingredient candidate substances used previously, xanthoangelol (Angelica keiskei) and quercetin 3-O-malonylglucoside (leaves of mulberry) were used as the indicator ingredients.

Extraction conditions, hot water extraction results, and alcohol extraction results are shown in Tables 1 to 3 below.

TABLE 1

| Hot water extraction conditions | | | Alcohol extractions conditions | | |
|---|---|---|---|---|---|
| Raw material input amount: | 500 | g | Raw material input amount: | 500 | g |
| Purified water input amount: | 10 L | | 40% EtOH input amount: | 10 L | |
| | (multiples of 20) | | | (multiples of 20) | |
| Temperature: | 110° | C. | Temperature: | 55° | C. |

| | | | |
|---|---|---|---|
| Time: | 1 h to 3.5 h | Time: | 2 h to 6 h |

TABLE 2
110° C. hot water extraction

| | Angelica keiskei | Morus alba L. |
|---|---|---|
| | Xanthoangelol | Quercetin 3-O-malonylglucoside |
| Time | (mg/kg) | (mg/kg) |

| | | | |
|---|---|---|---|
| 2 | h | 0.083 | 4.041 |
| 2.5 | h | 0.094 | 3.309 |
| 3 | h | 0.043 | 3.297 |
| 3.5 | h | 0.048 | 3.218 |

* In the case of Angelica keiskei extract, the content of indicator ingredients was low, so the analysis was performed after a 5-fold concentration.

TABLE 3
55° C. 40% alcohol extraction

| | Angelica keiskei | Morus alba L. |
|---|---|---|
| | Xanthoangelol | Quercetin 3-O-malonylglucoside |
| Time | (mg/kg) | (mg/kg) |
| 2 h | 27.202 | 85.504 |
| 4 h | 31.788 | 89.972 |
| 6 h | 38.042 | 89.385 |

From the results of Tables 1 to 3, it was confirmed that compared to the content in the case of hot water extraction, the content of indicator ingredient in Angelica keiskei was hundreds of times higher when Angelica keiskei was extracted with 40% alcohol, and the content of indicator ingredient in the leaves of mulberry was found to be about 26 to 28 times higher when the leaves of mulberry was extracted with 40% alcohol. From there, it was confirmed that the optimal extraction conditions for Angelica keiskei and the leaves of mulberry were 40% alcohol, a temperature of 55° C., and 4 hours of extraction.

### 1-2 Confirmation of Indicator Ingredients

To identify the concentrate powder content ratio of each of Angelica keiskei and leaves of mulberry relative to the raw material input ratio during composite extraction, a condition setting was required. To the end, Angelica keiskei and the leaves of mulberry were extracted under optimal extraction conditions (40% alcohol, a temperature of 60° C., 6 hours). Afterward, the obtainable solid weight, yield, and index content were confirmed. The results are shown in Tables 4 and 5 below.

TABLE 4

| Sample name | Solid content (powder) weight (g) | Yield |
|---|---|---|
| Angelica keiskei (20 g) | 2.8 g | 14% |
| Morus alba L. (20 g) | 0.7 g | 3.5% |

TABLE 5

| | | Detection |
|---|---|---|
| Raw material name | Indicator ingredient name | result (mg/g) |
| Angelica keiskei | Xanthoangelol | 1.57 |
| Morus alba L. | Quercetin 3-O-malonylglucoside | 10.3 |

As shown in Tables 4 and 5 above, relative to raw material input, the yield of concentrate powder obtained was 14% for Angelica keiskei and 3.5% for the leaves of mulberry, which meant that the yield in the case of Angelica keiskei was 4 times higher than that in the case of the leaves of mulberry, and Angelica keiskei and the leaves of mulberry included an indicator ingredient.

### <Example 2> Efficacy Analysis 1 of Composite Extract

### 2-1 Materials and Methods

A single extract of Angelica keiskei and a composite extract of Angelica keiskei and leaves of mulberry obtained under optimal extraction conditions (40% alcohol, 60° C., and 6 hours) according to Example 1-1 were prepared. The extracts were mixed in the ratios shown in Table 6 to form a composite (extract), and then the efficacy of the extracts depending on the ratios was compared.

TABLE 6

| Extract | | Ratio | Groups |
|---|---|---|---|
| Single extract | leaves of mulberry (M. alba L.) | — | ML |
| | ashitaba (A. keiskei) | — | AK |
| Composite extract | M. alba L. + A. keiskei | 1:1 | M1 |
| | (ML:AK) | 1:2 | M2 |
| | | 2:1 | M3 |

### (1) Measurement of DPPH Radical Scavenging Ability

A DPPH radical scavenging activity measurement experiment was conducted by modifying the method of Bloiss et al. To measure the electron-donating ability of a sample to DPPH radicals, 100 ul of sample diluted to the appropriate concentration was added to 150 ul of 0.4 mM DPPH solution dissolved in methanol and the mixture was reacted at a temperature of 37° C. for 30 minutes. Afterward, the absorbance of the reaction product was measured at a wavelength of 518 nm by using a microplate reader (Molecular Devices, San Jose, CA, USA). Trolox was used as a positive control, and antioxidant activity was calculated according to the following equation.

DPPH radical scavenging activity (%)={1−(B−C)/A}*100
A: DPPH absorbance
B: Absorbance of DPPH and sample reaction solution
C: Absorbance of the sample itself

### (2) Measurement of ABTS Cationic Radical Scavenging Ability

An ABTS radical scavenging activity measurement experiment was conducted by applying the method of Re et al. (1999). 7 mM ABTS and 2.45 mM potassium persulfate were mixed at a ratio of 1:1 (v/v), and the mixture was reacted in the dark at room temperature for 24 hours. As a result, an ABTS solution in which radicals were formed was prepared. Immediately before the experiment, the ABTS solution was diluted to get the absorbance thereof at a wavelength of 734 nm to be 0.7±0.02, and then the ABTS solution was used. 190 μl of the ABTS solution (which reached the appropriate absorbance) and 10 μl of the sample (which was diluted to various concentrations) were added to a sample and the mixture was reacted at room temperature for 6 minutes. Afterward, the absorbance of the reaction product was measured at a wavelength of 734 nm by using a microplate reader. Trolox was used as a positive control, and the ABTS radical scavenging activity results were calculated according to the following equation.

ABTS radical scavenging activity (%)={1−(B−C)/A}*100
A: ABTS absorbance
B: Absorbance of ABTS and sample reaction solution
C: Absorbance of the sample itself

### (3) Laboratory Animals

In this experiment, 6-week-old Sprague-Dawley rats purchased from Koatech Co., Ltd. (Pyeongtaek, Korea) were acclimatized for one week at the animal breeding facility of Dong-eui University College of Oriental Medicine. During that period, general symptoms were observed and only rats with no abnormalities were selected. The selected rats were separated into groups by a random method based on body weight range and then used in an experiment. The environmental conditions of the breeding box during the experiment period were maintained with an indoor temperature of 23±3° C. and a relative humidity of 50±10%, a light: dark cycle adjusted to a 12-hour cycle, and an illumination level of 150 to 300 Lux. Less than 2 animals per breeding box were housed in polycarbonate cages. Animal experiments were priorly reviewed by the Institutional Animal Care and Use Committee and then conducted in accordance with the regulations of the Institutional Animal Care and Use Committee of Dong-eui University (Approval number: A2022-020).

### (4) Dosage and Method of Administration

Experimental animals were randomly divided into 6 groups, and dosages are presented in Table 2. To induce muscle atrophy, Dexamethasone (Dex, 600 μg/kg/day) was orally administered to rats in all groups except the control group for 10 days. In oral administration, the control group (C) received sterilized distilled water, the induction group (D) received sterilized distilled water, the MA group received 100 mg/kg of the extract of leaves of mulberry, and the AK group received 100 mg/kg of the extract of Angelica keiskei, the MIX group received 100 mg/kg of a composite extract, and the positive control (PC) group received 320 mg/kg of 3-hydroxy-3-methylbutyric acid.

TABLE 7
Organization of groups in animal experiment

| Groups | No. of animals | Induction | Administration |
|---|---|---|---|
| C | 10 | Vehicle | Sterilized water |
| D | 10 | Dexamethasone | Sterilized water |
| ML | 8 | (600 μg/kg body weight/day) | MA (100 mg/kg body weight/day) |
| AK | 8 | | AK (100 mg/kg body weight/day) |
| MIX | 8 | | Mixture (100 mg/kg body weight/day) |
| PC | 10 | | 3-Hydroxy-3-methylbutyric acid |
| | | | (320 mg/kg body weight/day) |

### (5) Measurement of Grip Strength

The grip strength of all groups was measured 1 day before the end of the experiment. After having SD rats in the groups grasp the bar of the grip strength meter with both front paws thereof, the SD rats' bodies were horizontally kept. Maintaining that position, the rats' tails were pulled at a constant speed, and the maximum strength was recorded until the rats let go of the bar. A total of 7 measurements were recorded, and after excluding the highest and lowest values, the average of the measured values was taken as the result.

### (6) Autopsy and Blood Collection

12 hours before the end of the experimental period, the rats were fasted, an autopsy was performed under anesthesia using ether, and blood was collected from the heart. After the blood collection, major organs such as the lung, thymus, heart, kidney, spleen, and liver were removed and weighed (data not shown). The soleus and gastrocnemius muscles of both legs were separated from the rats and the size and weight of the muscles were measured.

### (7) Histopathological Examination by Hematoxylin and Eosin (H & E) Staining

To analyze the cross-sectional area of the muscles in this experiment, the soleus and gastrocnemius muscles were sectioned in paraffin at a diameter of 4 μm and stained using hematoxylin and eosin Y. Afterward, cross-sections of the stained soleus and gastrocnemius muscles were observed using a microscope (Evos Auto 2, Thermo Fisher Scientific, Waltham, MA, USA). The area of the cross-sections was measured using the Celeste image analysis program (Thermo Fisher).

### 2-2 Result

(1) Efficacy of Composite (Extract) of Leaves of Mulberry and Angelica keiskei on DPPH Radical Scavenging Activity

DPPH radicals are purple-colored free radicals. When the DPPH radicals react with antioxidants, the DPPH radicals are reduced, resulting in a decrease in the DPPH radicals and a color change of the radicals to yellow. Because of these properties, the DPPH radicals are known as an indicator for measuring the antioxidant activity of a sample.

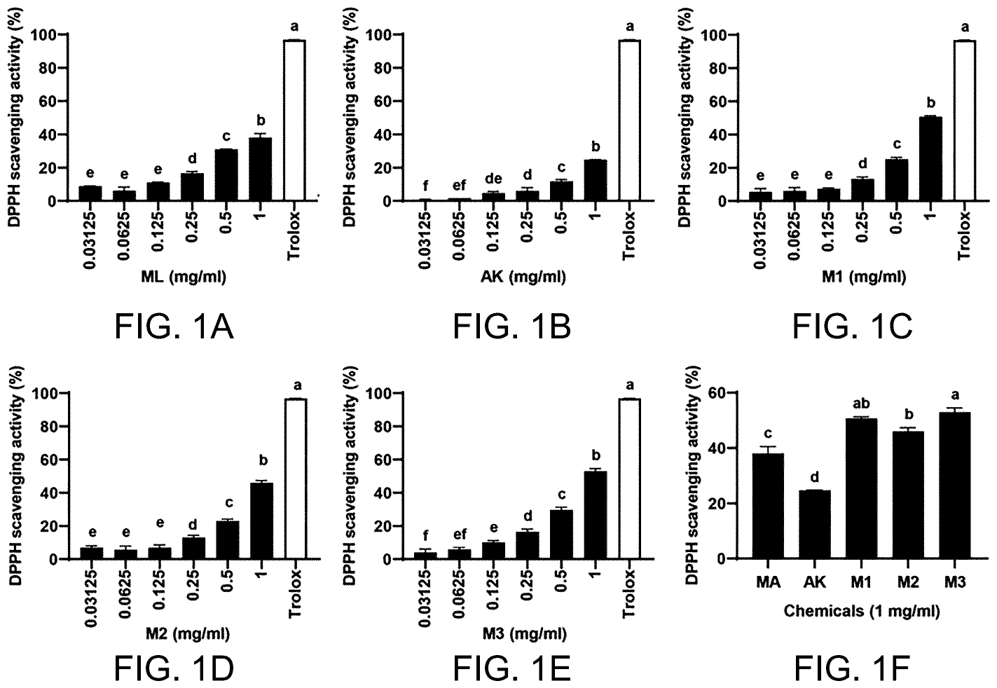

As shown in FIG. 1, DPPH radical scavenging activity significantly increased corresponding to an increased concentration of single extracts and composite extracts. In other words, at the highest extract concentration, 1 mg/ml, the DPPH scavenging activity level of the samples was highest at 52.92% for M3, which was a composite extract with a high proportion of leaves of mulberry, followed by M1 at 50.68% and M2 at 45.97%. As a result, it was found that the composite extracts showed a better DPPH radical scavenging ability than the single extracts (leaves of mulberry: 38.04%, Angelica keiskei: 24.69%).

(2) Efficacy of Composite (Extract) of Leaves of Mulberry and Angelica keiskei on ABTS Cationic Radical Scavenging Activity

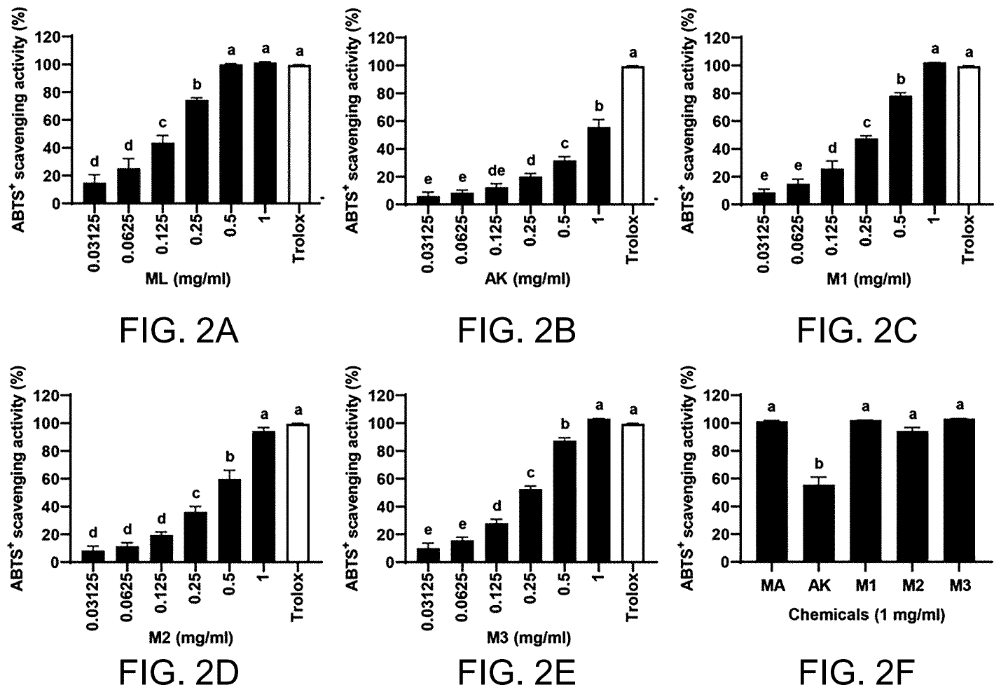

Antioxidants eliminate cation radicals generated by the reaction between ABTS and potassium persulfate, thereby blue-green ABTS solution turns colorless. The ABTS radical scavenging ability assay, herein, used the property of ABTS solution and measured the degree of colorlessness of the ABTS solution by absorbance. This method was different from the DPPH radical scavenging activity assay where DPPH scavenged free radicals. However, this method was a representative method for evaluating antioxidant activity along with the DPPH radical scavenging activity assay. The ABTS radical scavenging abilities of single extracts of each of the leaves of mulberry and Angelica keiskei and the ABTS radical scavenging abilities of composite extracts of the leaves of mulberry and Angelica keiskei mixed at various ratios were compared, and the comparison results are presented in FIG. 2. As a result of the experiments, it was observed that the radical scavenging abilities of samples were superior at high concentrations to those at low concentrations.

When comparing the ABTS cationic radical scavenging ability of each extract at a concentration of 1 mg/ml, M3 showed the highest ability level at 103.10%, followed by M1 at 102.08%, ML at 101.27%, M2 at 94.30%, and AK at 55.59%. Therefore, among single extracts of each of the leaves of mulberry and Angelica keiskei and composite extracts of the leaves of mulberry and Angelica keiskei, M3 was found to show high ABTS cationic radical scavenging activity.

Based on the results, it was confirmed that the antioxidant efficacy of M3 with a high ratio of the leaves of mulberry was superior to those of single extracts of each of the leaves of mulberry and Angelica keiskei. This suggested that M3 had the potential to become a material effective in various functionalities.

(3) Efficacy of Composite (Extract) of Leaves of Mulberry and Angelica keiskei on Dexamethasone-Induced Muscle Dysfunction in SD Rats.

Unlike the muscle atrophy model caused by immobilization, denervation, and disuse, a muscle atrophy model induced by dexamethasone is known to experience a decrease in Type II muscle fibers similar to sarcopenia and a resultant occurrence of myosin heavy chain transfer from fast type muscle fibers to slow type muscle fibers. Accordingly, in this example, animal models (rats) of muscle atrophy accompanied by dexamethasone-induced muscle dysfunction were made to determine whether M3 (MIX) and single extracts showed an effect in suppressing muscle atrophy, M3 having showed the best antioxidant activity in the cell-free system.

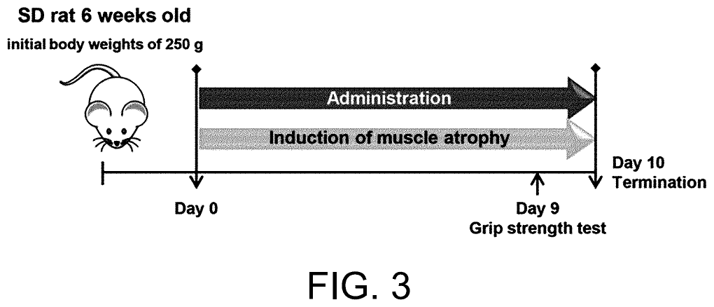

As shown in FIG. 3, 600 μg/kg of Dex and 100 mg/kg of single extracts and composite extracts were orally administered to the rats, the models' body weight was measured every two days, and grip strength was measured the day before autopsy. As a result, the body weight of the models decreased significantly compared to the control group from 2 days after administration of Dex to the end of the experiment. The ML, AK, MIX, and PC groups maintained their body weight similar to the induction group. Whereas, having the rats grasp the bar of the grip strength meter, the SD rats' bodies were horizontally kept. By pulling the rats back by their tail, the maximum grip strength of the rats was measured, thereby the actual muscle function of the rats was confirmed.

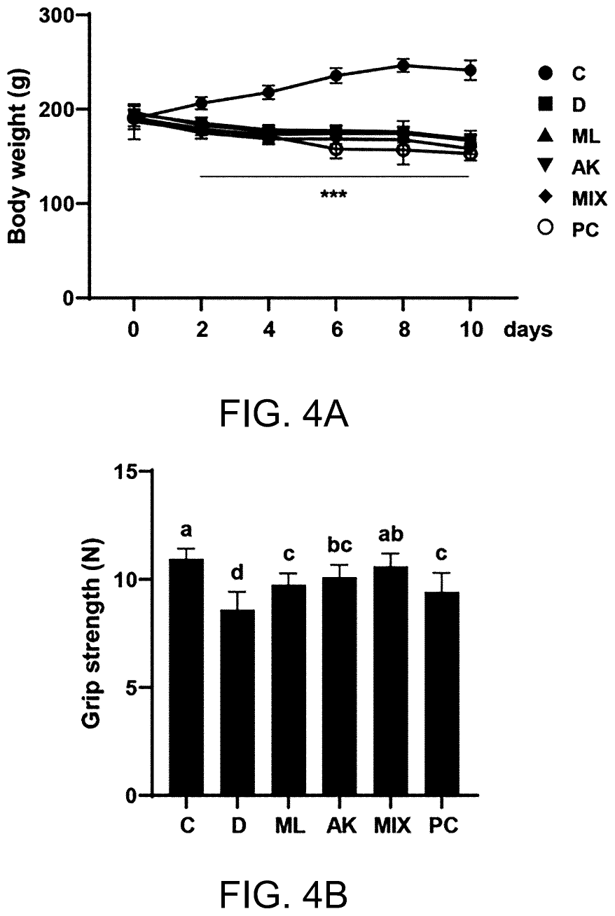

As shown in B of FIG. 4, compared to the control group the strength of which was 10.96±0.47N, the strength of the induction group decreased to 8.59±0.83N. Meanwhile, the strength of the ML and AK groups was 9.75±0.52N and 10.10±0.57N, respectively, which meant the strength of the two groups significantly increased compared to the induction group. In particular, the strength of the MIX group was 10.61±0.59N being recovered to the level of the control group.

(4) Efficacy of Composite (Extract) of Leaves of Mulberry and Angelica keiskei on Thickness and Weight of SD Rats' Soleus Muscle Reduced by Dexamethasone

Changes in the thickness and weight of the soleus muscle following 10 days of administration are shown in FIG. 5.

First, the thickness and weight of the soleus muscle were significantly decreased in the induction group (4.78±0.17 mm, 102±5.2 mg) compared to the control group (6.25±0.42 mm, 130.2±4.1 mg). The ML group (5.32±0.37 mm, 106.3±6.4 mg) and the AK group (5.33±0.24 mm, 104.5±7.5 mg) showed no significant difference in the thickness and weight from the induction group, but compared to the induction group, the MIX group (5.95±0.14 mm, 116.7 mm)±7.3 mg) showed a significant increase in the thickness and weight.

(5) Efficacy of Composite (Extract) of Leaves of Mulberry and Angelica keiskei on Dexamethasone-Induced Histological Changes in the Gastrocnemius Muscle of SD Rats

Typical properties of muscle atrophy are decreases in the size and number of muscle fibers. Based on that, histological changes through H & E staining of gastrocnemius muscle tissue in Dex-induced muscle atrophy models were confirmed.

As shown in FIG. 6A, compared to the control group, muscle fibers in the induction group were atrophied, resulting in decreases in the size of muscle fibers and a widened gap between muscle fibers. As a result, when quantitatively measuring the muscle fiber areas of the gastrocnemius cross-sections (FIG. 6B), the induction group (7.73±0.39 mm2) showed a significant difference from the control group (10.26±0.12 mm2). The areas of the ML group and AK group were 8.99±0.45 mm2 and 8.77±0.82 mm2, respectively, and the area of the MIX group was 9.57±0.41 mm2, indicating the best efficacy of the composite extract among the extract administration groups.

### <Example 3> Efficacy Analysis 2 of Composite Extracts

### Changes in the Expression of Factors for Muscle Differentiation and Muscle Atrophy Caused by Dexamethasone in SD Rats

In the process of treating muscle atrophy, the effects of the composite (extracts) of leaves of mulberry and Angelica keiskei on the expression of MyoD (a factor involved in muscle production) and on the expression of MuRF1 (a factor related to muscle atrophy) were confirmed. The expression of MyoD was decreased by dexamethasone meanwhile, the expression thereof was partially increased in the ML and AK groups.

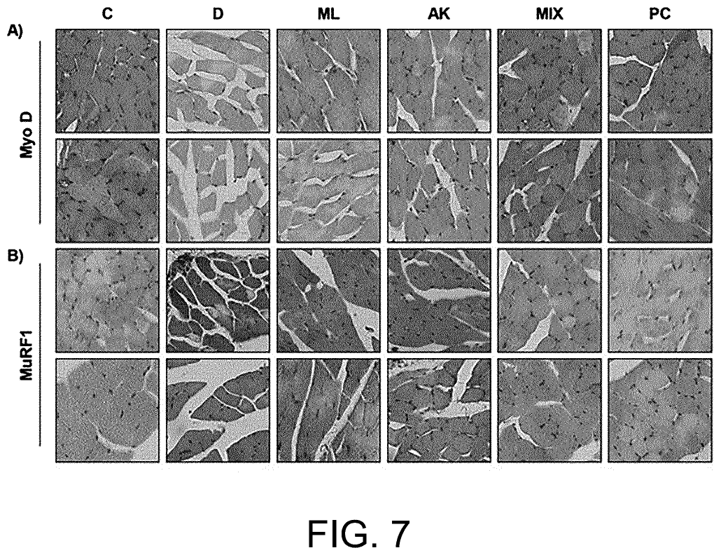

In particular, it was observed that the expression of MyoD was restored in the MIX group to a similar extent as those in the control and PC groups. The expression of MuRF1 (a factor involved in decomposing muscle proteins) increased in the dexamethasone-treated group compared to the control group. Meanwhile, the expression of MuRF1, unlike the increase in the expression induced by dexamethasone, tended to decrease in the MIX group and PC group (FIG. 7).

Therefore, the single extracts of each of the leaves of mulberry and Angelica keiskei and the composite extract of the two raw materials were believed to have a positive effect on treating muscle atrophy by promoting muscle production and controlling the mechanism of suppressing muscle protein breakdown.

### <Example 4> Establishment of Mixing Ratio and Optimal Extraction Conditions of Angelica keiskei and Leaves of Mulberry

The evaluation of the extract efficacy through cell experiments was conducted by mixing the concentrate powders of Angelica keiskei and leaves of mulberry in predetermined ratios (1:1, 2:1, 1:2), and a result that the powder mixture in a ratio of 1:2 showed the efficacy most was obtained in Example 2. Based on the result from Example 2, when extraction was conducted to obtain composite extracts, the mixing ratio of the raw materials was calculated using individual extract powder acquisition yield for Angelica keiskei and the leaves of mulberry As a result, it was confirmed that when the raw materials were mixed and added in the ratio of [Angelica keiskei: leaves of mulberry (8:1)], a powder mixture in the ratio of [Angelica keiskei concentrate powder: leaves of mulberry concentrate powder (1:2)] could be obtained.

After testing based on the extraction conditions obtained in Example 1, a 38.5% yield was obtained based on 23.5 Brix of a concentrate. In addition, when indicator ingredient content was analyzed by drying and powdering the concentrate, the content of xanthoangelol and quercetin 3-O-malonylglucoside was confirmed to be 0.51 mg/g and 5.46 mg/g, respectively. That meant that the content of xanthoangelol and quercetin 3-O-malonylglucoside was 97% and 80% consistent with the theoretical values of 0.523 mg/g and 6.87 mg/g, respectively. Extraction conditions, concentration results, and indicator ingredient content results are shown in Tables 8 and 9 below.

TABLE 8
Extractions conditions

| Raw material input | Angelica keiskei (0.3 kg):Morus | |
|---|---|---|
| amount: | alba L. (2.4 kg) | |
| 40% EtOH input | Multiple of 20 | |
| amount: | | |
| Temperature: | 55° | C. |
| Time: | 6 | h |

Concentration results

| | |
|---|---|
| Final Brix: | 23.5 |
| Final concentrate | 1,039 |
| volume (g): | |
| Concentration yield | 38.48% |
| (%): | |

TABLE 9

| | | Detection |
|---|---|---|
| Raw material name | Standard substance name | result (mg/g) |
| Angelica keiskei | Xanthoangelol | 0.51 |
| Morus alba L. | Quercetin 3-O-malonylglucoside | 5.46 |

Since the specific parts of the present disclosure have been described in detail above, various modifications and variations will be possible to those skilled in the art without departing from the essential characteristics of the present disclosure. Accordingly, the modified embodiments of the present disclosure are not intended to limit the technical idea of the present disclosure but are for explanation, and the scope of the technical idea of the present disclosure is not limited by these examples. The scope of protection of the present disclosure should be interpreted in accordance with the scope of the patent claims, and all technical ideas within the equivalent scope should be interpreted as being included in the scope of rights of the present disclosure.

1. A muscle strength-enhancing food composition comprising a composite extract of Angelica keiskei and leaves of mulberry as an active ingredient, the composite extract being obtained by mixing Angelica keiskei and the leaves of mulberry at a weight ratio of 1:8 to 16 and extracting the resulting mixture with alcohol,
the food composition promoting antioxidant activity (for example, DPPH scavenging activity and ABTS scavenging activity) and muscle function enhancement activity which suppresses muscle protein breakdown and promotes muscle building, whereby the food composition prevents a muscle-related disease by enabling a muscle to be maintained or strengthened.

2. The food composition of claim 1, wherein the muscle-related disease comprises at least one selected from the group consisting of atony, muscular atrophy, muscular dystrophy, muscle degeneration, muscle rigidity, atrophic axonal sclerosis, myasthenia gravis, cachexia, and sarcopenia.

3. The food composition of claim 1, wherein the composite extract contains xanthoangelol or 4-hydroxyderricin derived from Angelica keiskei as an active ingredient and quercetin, quercetin-3-O-malonyl glucoside, or chlorogenic acid derived from leaves of mulberry as an active ingredient.
